(19) United States
(12) Patent Application Publication
(10) Pub. No.: US 2016/0349188 A1
(43) Pub. Date: Dec. 1, 2016

(54) MICROFLUIDIC DEVICE FOR ANALYSIS OF FLOWING POLLUTANTS

(71) Applicants: CENTER NATIONAL DE LA RECHERCHE SCIENTIFIQUE (CNRS), Paris (FR); UNIVERSITÉ DE STRASBOURG, Strasbourg (FR)

(72) Inventors: Stephane LE CALVE, ROHR (FR); Alaa El Dine ALLOUCH, STRASBOURG (FR); Pierre BERNHARDT, HEILIGENBERG (FR); Maud GUGLIELMINO, WASSELONNE (FR); Christophe SERRA, SOUFFELWEYERSHEIM (FR)

(21) Appl. No.: 15/111,635

(22) PCT Filed: Jan. 14, 2015
(86) PCT No.: PCT/FR2015/050089

§ 371 Date: Jul. 14, 2016

(30) Foreign Application Priority Data
Jan. 14, 2014 (FR) 1450294
Feb. 13, 2014 (FR) 1451114

(51) Int. Cl. G01N 21/85; G01N 33/00; G01N 21/64; B01L 3/00; G01N 21/78

(52) U.S. Cl. CPC G01N 21/85; B01L 3/502753; B01L 3/50273; G01N 21/783; G01N 21/6428; G01N 33/0047; B01L 2300/0838; B01L 2300/18; B01L 2300/0654; G01N 2021/7766

(57) ABSTRACT

A microfluidic device for analyzing a gaseous compound, in a dynamic manner and while flowing, the device including mixing elements that make it possible to co-elute in a capillary tube a gaseous phase including the gaseous compound and a solution including a derivative agent; elements for eliminating the gaseous phase; and elements for determining the concentration of a gaseous compound. A method for determining the concentration of a gaseous compound using the microfluidic device is also described.

### FIELD OF THE INVENTION

This invention relates to a microfluidic device for trapping and for detecting that makes it possible to trap in a solution a gaseous compound soluble in said solution then to have it react with a derivative agent in order to form a species that can easily be detected and quantified, for example by colorimetry or fluorimetry. The invention also relates to a method of trapping and of detecting a gaseous compound using such devices.

### PRIOR ART

The device of this invention is particularly intended for analysing gaseous pollutants, such as formaldehyde, soluble in a solution, such as an aqueous solution.

Formaldehyde is present all throughout our environment. In the outdoor environment, it can come directly from industrial or automobile discharges or from forest fires or indirectly by oxidation of volatile organic compounds. Because it is highly soluble in water, formaldehyde can be found in the oceans, surface water or rainwater. Formaldehyde is also one of the major pollutants of indoor air, it is given off among others by paints, resins, treated wood, treated papers, textiles or by tobacco smoke. It is generally present in the indoor environment at concentrations typically varying between 10 and 100 μg/m³ (the concentration indoors is typically 2 to 15 times higher than the concentration outdoors). In the professional environment, its concentrations can reach several hundreds of μg/m³.

The impact on health of formaldehyde is increasingly taken into account by the public authorities. The French Agency for Food, Environmental and Occupational Health Safety has set a threshold limit of 30 μg/m³ which must not be exceeded in public-access buildings starting in 2015. This threshold will become more severe at 10 μg/m³ starting in 2022. The International Agency for Research on Cancer also modified the classification of formaldehyde in 2006, as such moving it from the category probable human carcinogen to the category human carcinogen.

A selective and accurate measurement of the emission rates of materials (in μg·m⁻²h-1) and of the concentrations in the air (μg/m³) appears therefore as necessary and essential.

Several devices for analysing formaldehyde have already been developed (A. Allouch et al., Transportable, fast and high sensitive near real-time analyzers: Formaldehyde detection, Sensors and actuators, B 181 (2013) 551-558). These devices generally include two successive steps: a step of trapping the formaldehyde and a step of detecting. Literature describes two methods of trapping formaldehyde. A first method is based on a trapping cell comprising a sensitive surface that includes an adsorbent (for example porous cellulose comprising a silica gel) and which has a change in the colour of the surface according to the adsorption of the formaldehyde. This method allows for substantial selectivity and sensitivity but is severely limited by its irreversibility and its discontinuous nature. A second method consists in transferring the gaseous formaldehyde to an aqueous solution. A reagent is then mixed with the solution enriched with formaldehyde in order to form a species that can be detected easily. Regardless of the method of trapping, the step of detecting is then carried out primarily using chromatographic, spectroscopic or chemical methods associated with fluorimetry or colorimetry.

International application WO 2010/142908 describes a device for determining the concentration of gaseous formaldehyde in a dynamic manner and while flowing comprising (1) an air pump in order to pump a predetermined quantity of gaseous phase comprising the formaldehyde, (2) means for transferring formaldehyde to an inert aqueous solution and (3) means for determining the concentration in aqueous phase of the formaldehyde. According to a first embodiment, the means for transferring include a capillary and a microporous tube. The gaseous phase comprising formaldehyde is injected jointly with an inert aqueous solution (here, water) in a capillary tube. The droplets that are formed in the capillary are co-eluted with the formaldehyde contained in the gaseous phase to a microporous tube which allows the gaseous phase to escape.

According to a second embodiment, the means for transferring include a microporous tube wherein an inert aqueous solution (here, an aqueous solution of nitric acid or water) circulates. The gaseous phase comprising formaldehyde then flows coaxially, outside of and around the microporous tube. The gaseous formaldehyde is then transferred to the inert aqueous solution which is located inside the microporous tube.

The means for determining the concentration in aqueous phase of the formaldehyde then include mixing means adapted to mix fluoral-p with on the one hand a substance for calibration and on the other hand a predetermined quantity of the aqueous phase enriched with formaldehyde during the preceding step. The fluoral-p then reacts with the formaldehyde in order to form a derivative compound (3,5-diacetyl-1, 4-dihydrolutidine: DDL) which can be detected, via a fluorescence cell, by fluorescence spectroscopy.

Disadvantages further limit the interest of this device, in particular the obstruction of the transfer module by clogging of the microporous tube, the substantial consumption of reagent of about 1 mL/min or the presence of at least one calibration substance of which the concentration of formaldehyde is known.

One of the objectives of this invention resides in the development of a microfluidic device that is reliable, accurate, sensitive, autonomous and which can be transported easily.

In order to achieve these objectives, it appears important to be able to ensure the reliability of the transfer module, limit the consumption of reagent, but also ensure the trapping and the derivatization reaction at the same location in order to lower the pitch of time between two measurements while still avoiding the use of additional substances.

In order to achieve the objectives of this invention, it is also necessary to associate a detection cell with the microfluidic device that offers better sensitivity and a faster renewal time that makes it possible to use limited liquid flow rates and therefore to save reagents.

### SUMMARY

The invention therefore relates to a microfluidic device for analysing a gaseous compound, in a dynamic manner and while flowing, said device comprising:
mixing means that make it possible to co-elute in a capillary tube a gaseous phase comprising said gaseous compound and a solution comprising a derivative agent;
means for reacting the gaseous compound in solution with the derivative agent;
means for eliminating the gaseous phase; and;
means for determining the concentration of a gaseous compound.

According to an embodiment, the mixing means, that make it possible to co-elute in a capillary tube a gaseous phase comprising said gaseous compound and a solution comprising a derivative agent, comprise a trapping cell comprising a capillary tube supplied with gaseous phase by a capillary gaseous phase supply tube and supplied with solution by a solution supply tube.

According to an embodiment, the means for reacting the gaseous compound in solution with the derivative agent comprise a means of thermoregulation that make it possible to regulate the temperature of the mixture between the solution comprising a derivative agent and the gaseous compound initially in gaseous phase trapped in the solution comprising a derivative agent.

According to an embodiment, the mixing means that make it possible to co-elute in a capillary tube a gaseous phase comprising said gaseous compound and a solution comprising a derivative agent, and the means for reacting the gaseous compound in solution with the derivative agent, are separated.

According to an embodiment, the mixing means that make it possible to co-elute in a capillary tube a gaseous phase comprising said gaseous compound and a solution comprising a derivative agent, and the means for reacting the gaseous compound in solution with the derivative agent, are combined.

According to an embodiment, the mixing means further comprise means for obtaining an annular flow, an annular slug flow or a slug flow.

According to an embodiment, the means for obtaining an annular flow, an annular-slug flow or a slug flow include a mass flow regulator adapted for gases, a gas pump and a peristaltic pump or a syringe pump regulated in such a way that the gaseous flow rate is 100 to 10,000 times greater than the liquid flow rate.

According to an embodiment, the means for obtaining an annular flow include a mass flow regulator adapted for gases, a gas pump and a peristaltic pump or a syringe pump regulated in such a way that the gaseous flow rate is 850 to 10,000 times greater than the liquid flow rate.

According to an embodiment, the capillary gaseous phase supply tube and the solution supply tube are perpendicular, concentric and oriented with respect to each other with an angle between 0° and 90°, preferentially between 0° and 60°.

According to an embodiment, said mixing means include a capillary tube supplied at its centre by a gaseous phase comprising said gaseous compound and at the periphery by the solution comprising a derivative agent.

According to an embodiment, said capillary tube of the mixing means is made from a hydrophilic material.

According to an embodiment, said capillary gaseous phase supply tube is of a smaller outer diameter than the inner diameter of the capillary tube, said capillary tube being inserted partially into the capillary tube of the mixing means.

According to an embodiment, the supplying with solution of the capillary tube of the mixing means by the solution supply tube is carried out upstream of the distal end of the capillary gaseous phase supply tube inserted partially into the first capillary tube of the mixing means.

According to an embodiment, the gaseous flow rate is 100 to 10,000 times greater than the liquid flow rate in order to obtain an annular flow.

According to an embodiment, said means for determining the concentration of a gaseous compound include:
means for measuring the concentration of the derivative compound obtained from the reaction between the gaseous compound and the derivative agent; and;
means for calculating the concentration of a gaseous compound using the concentration of the derivative compound obtained hereinabove.

According to an embodiment, the measurement of the concentration of the derivative compound is carried out by colorimetry. In this embodiment, the means for measuring the concentration of the derivative compound include a wave guide, a light source and a detector, such as a spectrometer.

According to an embodiment, the measurement of the concentration of the derivative compound is carried out by fluorescence spectroscopy. In this embodiment, the means for measuring the concentration of the derivative compound include a light source, a fluorescence cell comprising a wave guide and a detector, such as a photomultiplier.

According to an embodiment, the measurement of the concentration of the derivative compound is carried out by fluorescence spectroscopy and colorimetry.

The invention also relates to a method for determining the concentration of a gaseous compound using said device.

According to an embodiment, the gaseous compound to be analysed is formaldehyde.

According to an embodiment, the temporal resolution of said device is less than 5 min.

According to an embodiment, the consumption of liquid derivative agent is less than 1 mL/min.

The invention further relates to a microfluidic device for analysing a compound in an aqueous phase, in a dynamic manner and while flowing, said device comprising:
mixing means making it possible to mix in a capillary tube an aqueous phase comprising said compound and a solution comprising a derivative agent;
means for reacting said compound in solution with the derivative agent (2);
means for eliminating the gaseous phase that appeared during the reaction between said compound and the derivative agent; and;
means for determining the concentration of said compound.

The invention also relates to a method for determining the concentration of a compound in an aqueous phase using said device.

### DEFINITIONS

In this invention, the terms hereinbelow are defined in the following way:

“Derivative agent” designates a reagent reacting with the gaseous compound to be analysed in order to form a compound, referred to as a derivative compound, that can be detected and quantified easily.

“Co-elution” refers to the co-injection and the coexistence of a gaseous phase and of a liquid phase in a tube.

“Annular flow” refers to the flow regime of a two-phase liquid-gas flow wherein the gaseous phase circulates at the core of the flow and the liquid phase completely covers the wall as such forming an annular film around the gaseous phase. The obtaining of an annular flow is a function of the inner diameter of the tube, of the temperature and of the flow rate of the liquid and gaseous phases. Without leaving the scope of the invention, one or several of these parameters can be adapted, in light of the teaching of this invention in order to obtain an annular flow.

“Annular-pocket flow” or “annular-slug flow” refers to the instable and intermittent flow regime of a two-phase liquid-gas flow wherein the gaseous phase circulates at the core of the flow and the liquid phase at the periphery of the gaseous phase. This type of flow further comprises partially a narrowing of the gas flow when the liquid phase no longer completely covers the wall. The liquid phase as such partially wets the wall and forms an annular film around the gaseous phase. This regime is a transient regime between a slug flow and an annular flow. As such, an annular flow can comprise by intermittence an annular slug flow. The obtaining of an annular-slug flow is a function of the inner diameter of the tube, of the temperature and of the flow rate of the liquid and gaseous phases. Without leaving the scope of the invention, one or several of these parameters can be adapted, in light of the teaching of this invention in order to obtain an annular-slug flow.

“Pocket flows” or “slug flows” refers to the flow regime of a two-phase liquid-gas flow comprising elongated pockets or plugs of gas surrounded by a liquid film. The obtaining of a slug flow is a function of the inner diameter of the tube, of the temperature and of the flow rate of the liquid and gaseous phases. Without leaving the scope of the invention, one or several of these parameters can be adapted, in light of the teaching of this invention in order to obtain a slug flow.

“Flow with bubbles” refers to the flow regime of a two-phase liquid-gas flow comprising bubbles dispersed in the liquid phase.

“Proximal or distal” refers to the direction of flow of the flow in the microfluidic device; as such proximal refers to a position close to the inlet of the flow in an element of the microfluidic device and distal refers to a position close to the outlet of the flow in said element.

“Capillary tube” designates, interchangeably, a tube or a channel engraved in a solid, intended for the circulation of fluids.

### BRIEF DESCRIPTION OF THE FIGURES

Other particularities and advantages shall appear clearly in the description which is given of them hereinafter, for the purposes of information and in no way limiting, in reference to the annexed drawings, wherein:

FIG. 1 diagrammatically shows the device for analysis according to an embodiment of this invention.

FIG. 2 diagrammatically shows the device for analysis according to a second embodiment of this invention.

FIG. 3 diagrammatically shows the device for analysis according to a third embodiment of this invention.

FIG. 4 diagrammatically shows a detection cell according to an embodiment of this invention.

FIG. 5 is a cross-section view of a trapping cell according to an embodiment of this invention.

The drawings of the figures are not to scale. It goes without saying that the scope of the invention is not limited to the embodiments more specially described and shown in reference to the annexed drawings; on the contrary it encompasses all of the alternatives of it.

### REFERENCES

1 Source of gaseous compound to be analysed;
2 Solution comprising a derivative agent;
2′ Inert solution without derivative agent;
2″ Solution of derivative agent;
3 Peristaltic pump or syringe pump;
4 Mass flow regulator for gas;
5 Gas pump;
6 Trapping cell;
7 Means for eliminating the gaseous phase—Microporous tube;
8 Means of thermoregulation—Oven;
9 Wave guide;
10 Waste flask;
11 Light source—Deuterium/Halogen Lamp or pulsed xenon lamp;
12 UV-Visible spectrometer;
13 Detection cell (fluorescence, colorimetry or colorimetry and fluorescence pairing);
14 Optical fibre;
15 Channel, tube or capillary tube;
16 Source of excitation for the fluorescence—LED;
17 Photomultiplier for detecting fluorescence;
18 Hydrophilic capillary tube of the trapping cell;
19 Capillary gaseous phase supply tube of the trapping cell;
20 Solution supply tube of the trapping cell.

### DETAILED DESCRIPTION

This invention relates to a microfluidic device for analysing a gaseous compound, in a dynamic manner and while flowing, that makes it possible to trap in a solution a soluble gaseous compound and to have it react with a derivative agent in order to form a species that can be detected by said device.

Said microfluidic device for analysis comprises:
mixing means of a gaseous phase comprising said gaseous compound and of a derivative agent in solution;
means for eliminating the gaseous phase; and;
means for determining the concentration of a gaseous compound.

According to an embodiment, the microfluidic device further comprises means for reacting the gaseous compound in solution with the derivative agent.

According to a detailed embodiment hereinafter, the mixing means include a trapping cell that make it possible to co-elute in a capillary tube a gaseous phase comprising said gaseous compound and a solution comprising a derivative agent.

According to a detailed embodiment hereinafter, the means for reacting the gaseous compound in solution with the derivative agent include means of thermoregulating fluids and in particular the solution comprising the derivative agent.

In an embodiment, the mixing means of a gaseous phase comprising said gaseous compound and of a derivative agent in solution and the means for reacting the gaseous compound in solution with the derivative agent are combined (i.e. implemented simultaneously). As such, in this embodiment, the trapping cell is thermoregulated.

In an alternative embodiment, the mixing means of a gaseous phase comprising said gaseous compound and of a derivative agent in solution and the means for reacting the gaseous compound in solution with the derivative agent are separated (i.e. implemented successively). As such, in this embodiment, the trapping cell is not thermoregulated.

According to an embodiment wherein the mixing means of a gaseous phase comprising said gaseous compound and of a derivative agent in solution and the means for reacting the gaseous compound in solution with the derivative agent are combined; the mixing means include means for obtaining an annular flow or an annular slug flow.

According to an embodiment wherein the mixing means of a gaseous phase comprising said gaseous compound and of a derivative agent in solution and the means for reacting the gaseous compound in solution with the derivative agent are separated; the mixing means include means for obtaining an annular flow, an annular-pocket flow, a pocket flow or a bubble flow.

According to the inventors, the obtaining of an annular flow and, to a lesser degree the obtaining of an annular-pocket flow, is particularly advantageous because it makes it possible, for a given trapping tube, to improve the trapping efficiency and as such the sensitivity of the device for analysing. Indeed, in flows with bubbles or pockets, the liquid phase is pushed by the gas bubbles and the linear speed of the gas is substantially equal to that of the liquid. In the case of an annular flow, the linear speed of the gas is much higher than that of the liquid. As such the residence time of the liquid in the trapping cell, will be much higher than that of the gas.

The obtaining of an annular or annular slug flow is therefore important in the case where the mixing means and the means for reacting the gaseous compound in solution with the derivative agent are combined, this in order to guarantee a compact device and reduced analysis time.

In the case where the mixing means of a gaseous phase comprising said gaseous compound and of a derivative agent in solution and the means for reacting the gaseous compound in solution with the derivative agent are separated; the size of the trapping cell can be increased, for example by using a capillary tube with a spiral, in order to increase the gas-liquid contact time and to guarantee an optimum trapping efficiency and as such the same analytical sensitivity regardless of the type of flow.

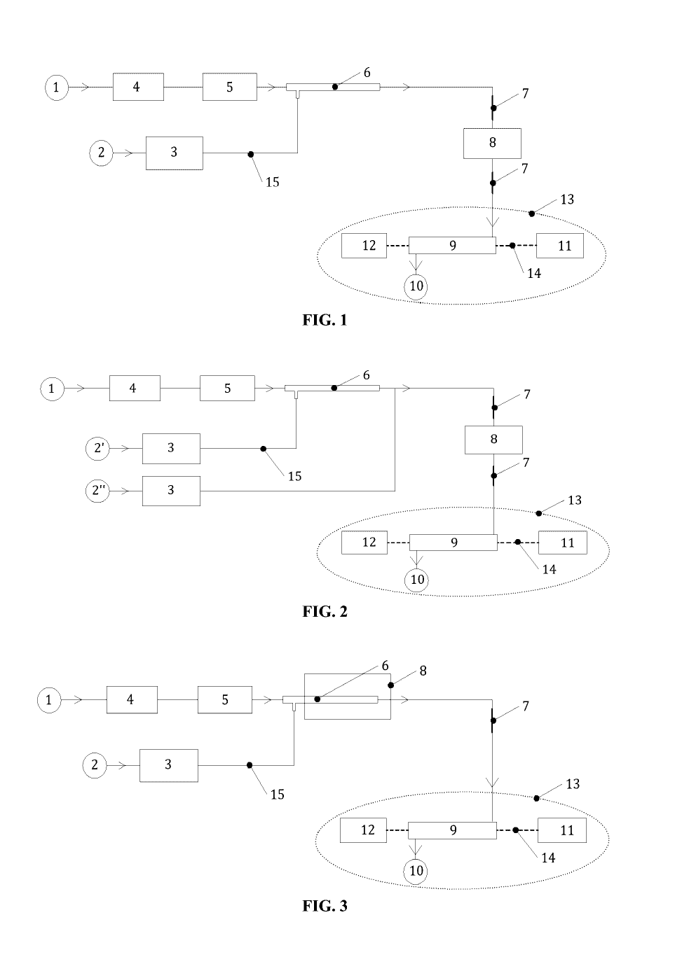

As shown in the three embodiments of FIGS. 1, 2 and 3, the mixing means of a gaseous phase comprising a gaseous compound to be analysed and a derivative agent in solution include at least one mass flow regulator adapted for gases 4, a gas pump 5 in order to carry out the transport at a perfectly stable flow rate of a gaseous phase (air or other gas) comprising the gaseous compound to be analysed, a solution, a peristaltic pump or a syringe pump 3 that carries out the continuous transport at a perfectly stable flow rate of said solution and a trapping cell 6. The gas pump 5 makes it possible to aspirate a predetermined quantity, using a mass flow regulator 4, of gaseous phase (air or other gas) comprising the gaseous compound to be analysed 1. According to an embodiment, the flow rate of the gas pump 5 is between 0.2 and 100 mL/min, preferentially between 1 and 50 mL/min, further more preferentially between 2 and 35 mL/min. According to an embodiment, capillary tubes 15 provide the transport of the gaseous phase to the trapping cell and have an inner diameter between 0.3 and 20 millimetres, more preferably between 0.7 to 8 millimetres. The peristaltic pump (or the syringe pump) 3 makes it possible to inject (or to push) a predetermined quantity of solution. According to an embodiment, the flow rate of the peristaltic pump 3 is between 0.1 and 100 μL/min, preferentially between 0.1 and 50 μL/min, further more preferentially between 0.2 and 35 μL/min. According to an embodiment, capillary tubes 15 provide the transport of the solution to the trapping cell and have an inner diameter between 0.2 and 2 millimetres, more preferably between 0.5 to 1 millimetre.

According to a preferred embodiment, shown in FIG. 1, the mixing means allow for the co-eluting, in a trapping cell 6, of a gaseous phase comprising said gaseous compound 1 and of a solution 2 comprising a derivative agent.

According to an alternative embodiment, shown in FIG. 2, the mixing means allow for the co-eluting, in a trapping cell 6, of a gaseous phase comprising said gaseous compound to be analysed 1 and an inert solution 2′. The derivative agent 2″ then being added downstream of the trapping cell 6. In this embodiment, the transport of the inert solution 2′ and of the derivative agent 2″ can be carried out using two peristaltic pumps (or syringe pumps) 3 or using a peristaltic pump (or syringe pump) 3 with at least two channels. A first channel supplies the trapping cell 6 with inert solution 2′ and a second channel supplies, downstream of the trapping cell, the solution enriched with the compound to be analysed, with derivative agent 2″.

The trapping cell 6 of the gaseous compound 1 is the location for the putting into contact of the gaseous compound to be analysed 1 with a solution 2 or 2′ in order to transfer the gaseous compound to be analysed into said solution. According to an embodiment of this invention, the trapping cell 6 carries out the trapping of the gaseous compound in a single cylindrical capillary tube 18 wherein are co-eluted the gaseous and liquid phases. Such a device avoids the risk of clogging of the pores of the trapping systems of prior art with two tubes, of which one is a microporous tube. The trapping cell 6 of this invention uses a liquid phase comprising directly 2 or not 2′ the derivative agent. According to an embodiment, the flow within the trapping cell 6 is a two-phase flow. According to an embodiment, the flow within the trapping cell 6 comprises at least one liquid phase (comprising directly 2 or not 2′ the derivative agent) and a gaseous phase (comprising the gaseous compound to be analysed 1). According to an embodiment, the trapping cell does not make it possible to generate multiple emulsions or second-order emulsions.

According to an embodiment, the mixing means make it possible to impose the orientation of the injection of fluids in relation to one another in the trapping cell 6. According to an embodiment, the trapping cell 6 comprises at least one means of injecting a gaseous phase into the capillary tube 18, such as a capillary gaseous phase injection tube 19. According to an embodiment, the trapping cell 6 comprises at least one means for injecting the liquid phase into the capillary tube 18, such as a liquid phase injection tube 20.

According to an embodiment, the orientation of the flows injected into the trapping cell depends on the arrangement of the gaseous and liquid phase supply tubes (19, 20).

According to an embodiment, the liquid phase 20 and gaseous phase 19 supply tubes are oriented perpendicularly. According to an embodiment, the liquid phase 20 and gaseous phase 19 supply tubes are oriented in parallel. According to an embodiment, the liquid phase 20 and gaseous phase 19 supply tubes are arranged concentrically. According to an embodiment, the liquid phase 20 and gaseous phase 19 supply tubes are oriented in such a way as to form a “Y”. According to an embodiment, the liquid phase 20 and gaseous phase 19 supply tubes are oriented with an angle between 0° and 90° with respect to one another, preferentially between 0 and 60°. According to an embodiment, the liquid phase 20 and gaseous phase 19 supply tubes are arranged at the same level in the trapping cell 6. According to an embodiment, the liquid phase 20 and gaseous phase 19 supply tubes are offset (i.e. are not arranged at the same height in the capillary tube 18) in the trapping cell 6. According to an embodiment, the liquid phase is injected on the periphery of the capillary tube 18 and the gaseous phase on the centre. According to an embodiment, the gaseous phase is injected on the periphery of the capillary tube and the liquid phase on centre.

According to an embodiment, the capillary tube 18, preferentially the inner surface of the cylindrical capillary tube 18 of the trapping cell, is hydrophilic.

According to the embodiment, as shown in FIG. 5, the gaseous compound is introduced at the centre of the cylindrical capillary tube 18 of the trapping cell 6 using a capillary gaseous phase supply tube 19, partially inserted at the centre of the trapping tube 18. A solution comprising 2 or not 2′ a derivative agent is inserted, using a capillary liquid phase supply tube 20, on the wall of the tube 18, for example using a tee fitting, at a height where the capillary tube 19 is always present. According to an embodiment, the capillary tube of the trapping cell 18 is hydrophilic, preferentially made of melted silica, in order to more easily obtain a flow of the annular type wherein the gaseous phase is located at the centre of the tube and the liquid phase on walls of the tube. According to an embodiment, the capillary gaseous phase supply tube 19 is preferentially made of melted silica. According to an embodiment, the annular flow regime of the liquid-gas mixture is established immediately at the injection of the fluids into the trapping cell 6. According to an embodiment, the annular flow regime of the liquid-gas mixture is delayed i.e. is not established immediately at the injection into the trapping cell 6 but during the circulation in the trapping cell 6.

According to an embodiment, in the trapping cell 6, in particular in the trapping tube 18, the ratio between the gaseous and liquid flow rates is between 100 and 10,000, preferentially between 500 and 5,000, further more preferentially 700 and 1,500. The degree of the gaseous flow rate in relation to the liquid flow rate advantageously makes it possible to reduce the consumption of solution.

According to an embodiment, in order to obtain an annular flow in the trapping cell, the ratio between the gaseous and liquid flow rates is between 850 and 10,000, preferentially between 1,000 and 5,000, further more preferentially between 1,000 and 3,500.

According to an embodiment, in order to obtain an annular-pocket flow in the trapping cell, the ratio between the gaseous and liquid flow rates is between 500 and 1,000, more preferentially between 600 and 850.

According to an embodiment, in order to obtain a pocket flow in the trapping cell, the ratio between the gaseous and liquid flow rates is between 100 and 600, preferentially between 125 and 550.

As such, for example, at ambient temperature and for an inner diameter of the trapping tube of 530 μm, with the device according to this invention, an annular flow is obtained for ratios of 1,000, 1,100, 1,200, 1,400, 1,500, 1,750, 2,000, 2,500, 3,000, or 3,500; an annular-pocket flow for ratios of 600, 750, 800, 850 and a pocket flow for ratios of 550, 500, 350, 250, 150.

Likewise, at 65° C. and for an inner diameter of the trapping tube of 530 μm, an annular flow is obtained for ratios of 850, 1,000, 1,400, 1,500, 2,000, 3,000, 4,000, 5,000; an annular-pocket flow for ratios of 550, 575, 650, 715, 750, 800; and a pocket flow for ratios of 150, 200, 250, 300, 400, 450, 500.

According to an embodiment, the trapping cell 6 does not contain a microporous tube 7.

According to an embodiment, the inner diameter of the hydrophilic capillary tube of the trapping cell 18 is between 100 and 1,000 μm, preferentially between 300 and 700 μm. According to an embodiment, the inner diameter of the hydrophilic capillary tube of the trapping cell 18 is 530 μm. The outer diameter of the capillary gaseous phase supply tube 19 is less than the inner diameter of the capillary tube 18. According to an embodiment, the outer diameter of the capillary gaseous phase supply tube 19 is between 50 and 850 μm, preferentially between 150 and 500 μm. According to an embodiment, the inner diameter of the capillary gaseous phase supply tube 19 is between 20 and 600 μm, preferentially between 50 and 300 μm, further more preferentially between 50 and 200 μm. The length of the hydrophilic capillary tube 18 of the trapping cell is adapted to allow for the total trapping of the compound to be analysed. For a given inner diameter, this parameter depends on the flow rate of the liquid phase: at 1 μL/min a length 10 times less is required than at 10 μL/min in order to obtain the same residence time of the liquid in the tube. As such according to an embodiment, the length of the capillary tube 18 can vary between 0.5 and 10 m, preferentially between 1 and 5 m for a liquid flow rate of 10 μL/min and between 5 cm and 1 m, preferentially between 10 and 40 cm for a liquid flow rate of 1 Note that the inner diameter of the hydrophilic capillary tube 18 also influences the thickness of the annular flow formed and therefore the residence time of the liquid for a given liquid flow rate, and in fact can modify the required length of the hydrophilic capillary tube 18. The length of the capillary gaseous phase supply tube 19 is adapted in order to allow for a sufficient gas flow rate according to Poiseuille's Law. According to an embodiment, the length of the capillary gaseous phase supply tube 19 is between 1 and 50 cm, preferentially between 2 and 20 cm. According to an embodiment, the capillary tube 19 is inserted into the capillary tube 18 in such a way that the external wall of the capillary tube 19 does not touch the internal wall of the capillary tube 18. According to an embodiment, the distance that separates the supply tube of the trapping cell in liquid phase 20 from the distal end of the capillary tube supplying the trapping cell with gaseous phase 19 is between 1 mm and 25 cm, preferentially between 1 mm and 10 cm.

According to an embodiment, the means for reacting the gaseous compound in solution with the derivative agent include means of thermoregulating fluids and in particular the solution. According to an embodiment, the microfluidic device of this invention comprises at least one means of thermoregulation 8. The step of thermoregulating that makes it possible to accelerate the kinetics of the reaction between the compound to be analysed and the derivative agent is carried out using a means of thermoregulation 8. According to an embodiment, the means of thermoregulation 8 is located downstream of the trapping cell 6. According to an embodiment, the means of thermoregulation 8 is located on the trapping cell 6. According to an embodiment, said means, is an oven, a heating resistance or any other means within the scope of those skilled in the art. According to an embodiment, the temperature of the means of thermoregulation 8 is between 20 and 150° C., preferentially between 40 and 130° C., further more preferentially between 50 and 80° C.

According to an embodiment, as shown in FIGS. 1 and 2, the gaseous flow is evacuated at the outlet of the trapping cell 6 using means for eliminating the gaseous phase 7. The capillary tube 15 comprising the solution of derivative agent and the compound to be analysed trapped in said solution is then arranged in a means of thermoregulation that make it possible to regulate the temperature to a value that favours the reaction between the derivative agent and the compound to be analysed. In a particular embodiment wherein the compound to be analysed is formaldehyde and the reagent used is fluoral-p, the means of thermoregulation 8, for example an oven can be at a temperature between 20 and 100° C., preferentially between 50 and 80° C., further more preferentially 65° C. The efficiency of the reaction depends on the residence time of the liquid mixture in the means of thermoregulation 8, which is a function of both the flow rate and of the volume of the capillary which itself depends on the length of the capillary and on its inner diameter. The capillary 15, arranged in the means of thermoregulation 8 can have a length between 0.02 and 10 m, preferentially between 0.05 and 5 m, further more preferentially between 0.10 and 2 m. In an embodiment, the residence time of the solution in the means of thermoregulation 8 is greater than 30 seconds, preferentially greater than 60 seconds; further more preferentially between 60 seconds and 5 minutes; in any case, the residence time of the solution in the means of thermoregulation 8 is less than 10 minutes, preferentially less than 5 minutes. In a particular embodiment wherein the compound to be analysed is formaldehyde and the reagent used is fluoral-p, the residence time of the solution in the means of thermoregulation 8 is preferentially greater than 2 minutes, further more preferentially greater than 3 minutes; in any case, the residence time of the solution in the means of thermoregulation 8 is less than 5 minutes.

According to an embodiment, shown in FIG. 3, the means of thermoregulation 8 is located on the trapping cell 6. This mode of trapping allows for the carrying out of the trapping and the reaction between the compound to be analysed and the derivative agent during the same step. As such, the response time of the measurement between the moment when the molecule of gaseous formaldehyde is trapped and the moment when it is detected is substantially reduced. The capillary tube 18 of the trapping cell is arranged in a means of thermoregulation 8 that make it possible to regulate the temperature to a value that favours the reaction between the derivative agent and the compound to be analysed. Such a trapping device heated, using means of thermoregulation 8, to a temperature higher than the ambient temperature advantageously prevents the condensation of water vapour around the microporous tube of the trapping devices of prior art. Indeed the air injected into the trapping device can have a high hygrometry and a change in the temperature between the ambient air and the trapping device can result in the formation of condensation that hinders the trapping of the gaseous compound to be analysed through the microporous tube and distorts the analysis. The presence of a heated trapping device as in this microfluidic device makes it possible to prevent this problem. In an embodiment wherein the means of thermoregulation 8 is located on the trapping cell 6, the capillary tubes 15 allowing for the aspiration and the transport of the gaseous phase to the trapping cell 6 are also heated using an additional means of thermoregulation, such as an oven, a heating resistance or any other means within the scope of those skilled in the art.

In a particular embodiment wherein the compound to be analysed is formaldehyde and the reagent used is fluoral-p, the means of thermoregulation 8, for example an oven, can be at a temperature between 20 and 100° C., preferentially between 50 and 80° C., further more preferentially 65° C. The gaseous flow is then evacuated at the outlet of the trapping cell 6 and of the means of thermoregulation 8 using means for eliminating the gaseous phase 7. In an embodiment, the residence time of the solution in the means of thermoregulation 8 is greater than 30 seconds, preferentially greater than 60 seconds, further more preferentially between 60 and 5 minutes; in any case, the residence time of the solution in the means of thermoregulation 8 is less than 10 minutes, preferentially less than 5 minutes. In a particular embodiment wherein the compound to be analysed is formaldehyde and the reagent used is fluoral-p, the residence time of the solution in the means of thermoregulation 8 is preferentially greater than 2 minutes, further more preferentially greater than 3 minutes; in any case, the residence time of the solution in the means of thermoregulation 8 is less than 5 minutes. This embodiment is made possible thanks to the annular flow in the trapping cell 6 which makes it possible to have a lower liquid flow rate, and therefore a residence time of the liquid that is sufficient so that the reaction between the compound to be analysed and the derivative agent is complete, while still limiting the length of the capillary tube 18.

According to an embodiment, an additional means of thermoregulation can be added between the means for eliminating the gaseous phase and the means for determining the concentration of a gaseous compound or between the means of thermoregulation 8 and the means for determining the concentration of a gaseous compound. This additional means of thermoregulation is preferentially a means of cooling so that the flowing solution is not at an excessively high temperature, preferentially at a temperature between 10 and 30° C., during the passing by the means for determining the concentration of a gaseous compound (i.e. during the passing by the detection cell).

According to an embodiment, the device according to this invention comprises means 7 for eliminating the gaseous phase. As such the gaseous phase comprising initially the gaseous compound to be analysed and/or the bubbles of air or of gas that appeared in the means of thermoregulation and/or the bubbles of air or of gas that appeared during the reaction between the compound to be analysed and the derivative agent do not disturb, in particular by not increasing the noise, the means for determining the concentration of a gaseous compound. The sensitivity and the accuracy of the measurement are as such improved. According to an embodiment, said means 7 are one or several microporous tubes that allow the gases to pass but not the liquids. According to an embodiment, said means 7 are made from any material within the scope of those skilled in the art that is inert and porous, such as for example microporous Teflon. In an embodiment, the microporous tube is a tube made of microporous Teflon. In an embodiment, the microporous tube measures from 2 to 10 cm long and 0.1 to 1.5 mm in inner diameter.

According to an embodiment, the means 7 for eliminating the gaseous phase are located between the trapping cell 6 and the means of thermoregulation 8 and/or between the means of thermoregulation 8 and the means for determining the concentration of a gaseous compound.

According to an embodiment, the means for determining the concentration of a gaseous compound include:
means for measuring the concentration of the derivative compound obtained from the reaction between the compound to be analysed and the derivative agent; and;
means for calculating the concentration of a gaseous compound using the concentration of the derivative compound obtained hereinabove.

The means for measuring the concentration of derivative compound can include any means for measuring known to those skilled in the art such as fluorimetry, colorimetry, mass spectrometry, etc.

According to an embodiment, as shown in FIGS. 1, 2 and 3, the means for measuring the concentration of the derivative compound include a detection cell 13 by colorimetry. In this embodiment, said cell comprises:
a light source 11, preferentially a Deuterium/Halogen lamp or a pulsed xenon lamp;
a wave guide 9, preferentially a wave guide with a liquid core, further more preferentially a wave guide with a liquid core made of Teflon AF2400; and;
a spectrometer 12, more preferably a UV-Visible spectrometer with a spectral resolution of 0.05 to 15 nm, further more preferentially a UV-Visible spectrometer with a spectral resolution of 1 to 12 nm.

In this embodiment, a first optical fibre 14 connects the light source to the wave guide and a second optical fibre 14 connects the wave guide to the UV-Visible spectrometer, in order to collect the light intensity and to limit any disorder concerning for example the alignment of the beams. According to an embodiment, the inner diameter of the wave guide is between 100 μm and 2 mm, preferentially between 200 and 1,000 μm.

According to an embodiment, the length of the wave guide is between 1 and 100 cm, preferentially between 5 and 90 cm, further more preferentially between 5 and 40 cm. Preferentially the residence time in the wave guide is less than 20 minutes, preferentially less than 5 minutes, further more preferentially less than 1.5 minutes in order to prevent photolysis of the derivative compound. To do this, the diameter of the wave guide can be modified and the liquid flow rate which, in the case of pairing with the microfluidic trapping cell 6, is preferentially between 0.1 and 100 μL/min, more preferentially between 1 and 50 μL/min, further more preferentially between 2 and 30 μL/min. In the case of a use of the colorimetric cell 9 without pairing with the microfluidic trapping cell 6, the flow rate can be increased up to 5 mL/min, preferentially up to 2 mL/min.

According to an embodiment, the detection cell by colorimetry can have SMA connectors (SubMiniature version A) or any other connectors for optical fibre within the scope of those skilled in the art, making it possible to connect on the one hand the wave guide to an optical fibre connected to a UV-Visible spectrometer, and on the other hand the wave guide to an optical fibre connected to a light source. According to an alternative embodiment, the light source and/or the UV-Visible spectrometer are attached to the wave guide, which makes it possible to overcome optical fibres. In this embodiment, a device for evacuating the heat of the light source can be added.

Beer-Lambert's Law makes it possible to connect the absorbance and the concentration of the derivative compound trapped in solution. Knowing the stoichiometry of the reaction between the compound to be analysed and the derivative agent and the trapping efficiency of the gaseous compound in solution (assumed to be equal to 100%), the concentration of a gaseous compound to be analysed can be known. In the case where the compound to be analysed is formaldehyde and the derivative agent is fluoral p, the derivative compound is DDL. As 1 mole of formaldehyde reacts with 2 moles of fluoral-p in excess in order to form 1 mole of DDL, the number of moles of formaldehyde experimentally trapped in solution using the absorbance measured can be calculated.

If the trapping of the gaseous formaldehyde is considered to be total, the gaseous formaldehyde is quantitatively transferred to an aqueous solution, which allows us to determine the number of moles of formaldehyde theoretically trapped in solution. The trapping efficiency is then the ratio between the number of moles of formaldehyde experimentally trapped and the theoretical number of moles of formaldehyde with a trapping of 100%. The trapping efficiency of the microfluidic device according to this invention, obtained for concentrations of gaseous formaldehyde varying between 20 and 160 μg/m³ was found equal to 88+/−12% regardless of the concentrations.

The conditions of this test were the following: liquid flow rate of 10 μL/min, gaseous flow rate of 10 mL/min, length of the trapping tube of 2.5 m, inner diameter of the trapping tube of 430 μm, temperature of the means of thermoregulation of 65° C. and residence time of the liquid of 3.3 min. In this test the mixing means of the gaseous phase comprising said gaseous compound and of the derivative agent in solution and the means for reacting the gaseous compound in solution with the derivative agent were combined (the trapping is carried out at 65° C.).

Other tests wherein the mixing means of the gaseous phase comprising said gaseous compound and of the derivative agent in solution and the means for reacting the gaseous compound in solution with the derivative agent are combined were carried out. The trapping efficiencies of the microfluidic device for inner diameters of the trapping cell of 530 μm, 430 μm and 320 μm are respectively 100+/−16%, 107+/−18% and 118+/−22%. In another test, wherein the mixing means of the gaseous phase comprising said gaseous compound and of the derivative agent in solution and the means for reacting the gaseous compound in solution with the derivative agent are separated (the trapping is carried out at ambient temperature), the trapping efficiency of the device was found to be equal to 90+/−2%. In this test, the liquid flow was 50 μL/min, the gas flow 50 mL/min, the length of the trapping tube 2 m, the inner diameter of the trapping tube 680 μm, the temperature of the means of thermoregulation 65° C. and the residence time of the liquid in the oven 4 min.

According to an embodiment (not shown), the means for measuring the concentration of the derivative compound include a detection cell by fluorimetry. In this embodiment, said cell comprises:
at least one source of excitation excitant the fluorescence of the derivative compound, preferentially at least one light-emitting diode (LED);
a wave guide, preferentially a wave guide with a liquid core, further more preferentially a wave guide with a liquid core made of Teflon AF2400; and;
a photomultiplier collecting the fluorescence of the derivative compound.

In this embodiment, a first optical fibre connects the source of excitation to the wave guide and a second optical fibre connects the wave guide to the photomultiplier, in order to collect the light intensity and to limit any disorder concerning for example the alignment of the beams. In the case where the derivative compound is DDL, the light source can be a LED emitting at 415+/−20 nm exciting the fluorescence of the DDL. According to an embodiment, a filter centred on the wave guide of the fluorescence to be measured is arranged in front of the photomultiplier in order to eliminate the parasite fluorescence and/or the parasite light emitted by the source of excitation and as such collect the fluorescence emitted only by the derivative compound. According to an embodiment, the source of excitation is placed in such a way that the angle between the source of excitation and the wave guide is between 90° and 180°, with a lighting in the direction opposite that of the flow of the solution, more preferentially between 100 and 160°, further more preferentially 120°. According to an embodiment, the inner diameter of the wave guide is between 100 μm and 2 mm, preferentially between 300 μm and 1 mm. According to an embodiment, the length of the wave guide is between 1 and 20 cm, preferentially between 2 and 10 cm, further more preferentially between 3 and 5 cm.

The wave guide transmits the excitation light and the light coming from the fluorescence of the molecules. The molecules of the derivative compound that are directly illuminated, but also those located upstream of the lighting, are excited since the wave guide makes it possible to propagate the excitation light. The fluorescence light, which is anisotropic in a conventional fluorescence system, is “trapped” in the wave guide of this invention, and propagates as such in both directions of the wave guide in probably similar proportions. As such, a large portion of the fluorescence, about 50%, is collected on the detector which is preferentially placed in the axis of the wave guide downstream of the light source so as to limit the number of photons emitted by the source which reach the detector. The sensitivity of the means of measurement by fluorimetry is as such greatly improved.

According to an embodiment, the volume of the microfluidic fluorescence cell is low and between 0.1 and 100 μL, preferentially between 0.15 μL and 35 μL. In a conventional detection cell made of quartz of parallelepiped shape the renewing of the cell by the flowing solution is significantly longer and the volume is greater for the same sensitivity.

According to an alternative embodiment, the detection cell by fluorimetry can comprise in place of the wave guide any type of fluorescence cell within the scope of those skilled in the art. According to an embodiment the inner diameter of the wave guide is between 0.05 mm and 5 mm, preferentially between 0.1 mm and 2 mm. According to an embodiment wherein the microfluidic fluorescence cell 9 is paired with the microfluidic trapping cell 6, the liquid flow rate is between 0.1 and 100 μL/min, preferentially between 1 and 50 μL/min, further more preferentially between 2 and 30 μL/min. In the case of a use of the fluorescence cell without pairing with the microfluidic trapping cell 6, the flow rate can be increased up to 5 mL/min, preferentially up to 2 mL/min.

According to an embodiment, the detection cell by fluorimetry can have SMA connectors (SubMiniature version A) or any other connectors for optical fibre within the scope of those skilled in the art, making it possible to connect on the one hand the wave guide to an optical fibre connected to a photomultiplier, and on the other hand the wave guide to an optical fibre connected to a source of excitation. According to an alternative embodiment, the source of excitation and/or the photomultiplier are attached to the wave guide, which makes it possible to overcome optical fibres. In this embodiment, a device for evacuating the heat of the source of excitation can be added. According to an embodiment, the detection cell comprises at least one source of excitation, more preferably 2, 3 or 4 sources of excitation in order to increase the excitation light and therefore the fluorescence emitted. According to an embodiment, the wall of the wave guide is hydrophobic in order to prevent the phenomena of adsorption of the derivative compound.

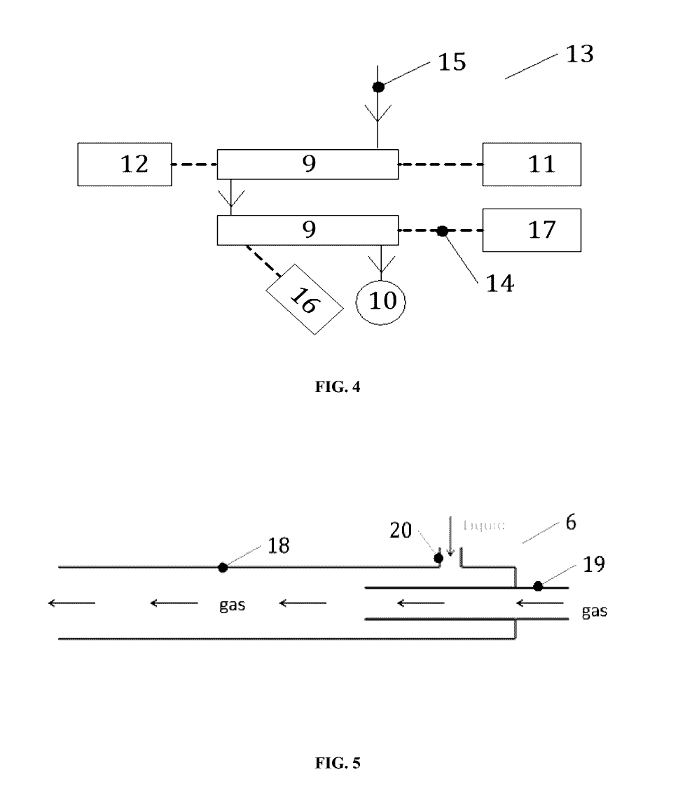

According to an embodiment, the means for measuring the concentration of the derivative compound include a detection cell by colorimetry coupled to a detection cell by fluorimetry. In this embodiment, shown in FIG. 4, the capillary tube 15 comprising the derivative compound passes through a first wave guide 9, illuminated by a light source 11 and the absolute concentration (by assuming that the trapping efficiency is 100%) of derivative compound is then determined using the spectrometer 12. As colorimetry is not destructive, the solution can then be passed in a second wave guide 9, illuminated by a source of excitation 16, the fluorescence of the molecules of the derivative compound excited by the source 16 is then collected using a photomultiplier 17. The solution is then collected in a waste flask 10.

In this embodiment, the device of this invention does not require any calibration substance because the calibration of the means of measurement by fluorimetry is carried out using the means of measurement by colorimetry. Indeed colorimetry is independent of the intensity of the light source which will drop with the ageing of the equipment, contrary to fluorimetry which requires daily calibration. In order to not require calibration substances, within this microfluidic device a system of colorimetry is coupled with a system of fluorimetry. The fluorimetry does however have an interest because it allows for the determination of lower concentrations. As such the detection cell by colorimetry makes it possible to measure low to high concentrations, typically 1 to 2,000 μg/m³ with a detection limit of 1 μg/m³ and the detection cell by fluorimetry makes it possible to measure very low to medium concentrations, typically 0.1 to 500 μg/m³ with a detection limit of 0.3 μg/m³. In this embodiment, a first optical fibre 14 connects the light source 11 to the first wave guide 9, a second optical fibre 14 connects the first wave guide 9 to the spectrometer 12, a third optical fibre 14 connects the source of excitation 16 to the second wave guide 9 and a fourth optical fibre 14 connects the second wave guide 9 to the photomultiplier 17. According to an alternative embodiment, the or said optical fibres 14 can be omitted and the light source 11 and/or, the spectrometer 12 and/or the source of excitation 16 and/or the photomultiplier 17 can be attached to the wave guides 9.

According to an embodiment, the means for calculating the concentration of a gaseous compound include an electronic or computerised device, such as a microcontroller or a computer, connected to the detector, namely the spectrometer or the photomultiplier, as such received the raw signal and calculating the concentration of the gaseous compound to be analysed in the gaseous phase. According to an embodiment, a computer interface controls the entire microfluidic device.

This invention also relates to a method for determining the concentration of a gaseous compound using the device of this invention.

According to an embodiment, the gaseous compound analysed by the microfluidic device according to this invention is preferentially a soluble gaseous pollutant, further more preferentially formaldehyde.

According to an embodiment, the microfluidic device according to this invention is used to determine the concentration in a gaseous phase of gaseous compounds that have a relatively high Henry constant (H), i.e. between 0.005 M/Pa (20 M/atm) and 2.96 M/Pa (3*10⁵ M/atm). Mention can be made by way of example of formaldehyde (H=0.03 M/Pa or 3,100 M/atm), methyl hydroperoxide and compounds of the same family (H=0.003 M/Pa or 310 M/atm), hydrogen peroxide (H=1.09 M/Pa or 1.1*10⁵ M/atm), glyoxal (H=2.96 M/Pa or 3.0*10⁵ M/atm), methyl glyoxal (H=0.32 M/Pa or 3.2*10⁴ M/atm), carboxylic acids (H>0.001 M/Pa or 1,000 M/atm) or phenol and derivatives thereof such as cresols (H>0.005 M/Pa or 500 M/atm).

According to an embodiment, the derivative agent used by the microfluidic device according to this invention can be any derivative agent that reacts specifically with the gaseous compound to be analysed in order to form a derivative compound that can be detected by conventional methods of detection (colorimetry, fluorimetry, etc.). Preferably, the derivative agent has both a quantum efficiency of fluorescence and a reaction speed with the compound to be analysed that are high. As such it has been shown that fluoral-p reacts preferentially with the formaldehyde in relation to the other aldehydes present in the air such as glyoxal, acetaldehyde or hexanal. At equal temperature and concentrations, the kinetics of the reaction between fluoral-p and formaldehyde is 3,000 to 10,000 times faster. In addition, it has also been shown that the quantum efficiency of derivative compounds obtained from the reaction between fluoral-p and the other aldehydes present in the air is substantially lower than the quantum efficiency of DDL.

According to an embodiment, the solution comprising a derivative agent 2 or the solution 2′ wherein is added a derivative agent 2″ downstream of the trapping cell 6, is any solution that makes it possible to solubilise the derivative agent and the compound to be analysed. According to an embodiment, said solution is preferentially:
an aqueous solution: pure water, aqueous solution of nitric acid, etc.;
a water-alcohol mixture such as a water/ethanol mixture with a ratio 90/10 (v/v);
an alcohol, such as ethanol or isopropanol.

Preferably, in the absence of derivative agent, the solution does not directly react with the compound to be analysed coming from the gaseous phase and this compound is perfectly soluble in said solution. According to an embodiment, a catalyst can be added to the solution in order to accelerate the reaction between the derivative agent and the gaseous compound to be analysed.

According to a preferred embodiment, the microfluidic device according to the invention is used to determine the concentration of the formaldehyde present in a gaseous phase with fluoral-p as derivative agent. The fluoral-p reacts specifically with the formaldehyde in order to form 3,5-diacetyl-1,4-dihydrolutidme (or DDL).

The microfluidic device according to this invention allows for a reduction in the consumption of solution 2 or 2′ and 2″ and allows for an increase in the autonomy of the device, in particular thanks to the use of an annular flow in the trapping cell. According to an embodiment, the consumption of solution is less than 1 mL/min, about 10 μL/min. As such with the device described in this invention, 10 mL of reagents are sufficient for carrying out nearly 500 analyses. For the purposes of comparison, the device described in international application WO 2010/142908 made it possible to carry out only a single analyse with the same quantity of reagent. The autonomy and the size of the device are substantially improved.

The microfluidic device according to this invention allows for a decrease in the temporal resolution of the device, in particular thanks to the carrying out of the trapping and the derivatization reaction in situ. According to an embodiment, the temporal resolution of this device is less than 5 minutes, preferentially less than 2 minutes, further more preferentially of about 1 minute. According to an embodiment, the initial response time of the device is less than 20 minutes, preferentially less than 10 minutes, further more preferentially of about 5 to 6 minutes.

According to an embodiment, the microfluidic device can be transported easily; it is made with means that are not very voluminous and light and requires few reagents 2 or 2′ and 2″. According to an embodiment, a microcontroller and/or a display device are integrated into the device according to this invention in order to guarantee a perfectly autonomous device.

According to an embodiment, the solution 2 or the solutions 2′ and 2″ are stored inside the microfluidic device in a flask or a microplate that is easy to replace once the solution is consumed.

According to an embodiment, all of the capillaries 15 of the device, except the capillary tube 18 of the trapping system 6 and the microporous tube(s) 7, are made of PEEK or any other material within the scope of those skilled in the art.

This invention also relates to a microfluidic device for analysing a compound in aqueous phase, in a dynamic manner and while flowing, making it possible to mix said compound with a solution comprising a derivative agent, and to have it react with said derivative agent in order to form a species that can be detected by said device.

Said microfluidic device for analysis comprises:
mixing means making it possible to mix in a capillary tube an aqueous phase comprising said compound and a solution comprising a derivative agent;
means for reacting said compound with the derivative agent;
means for eliminating the gaseous phase that appeared during the reaction between said compound and the derivative agent; and;
means for determining the concentration of said compound.

According to an embodiment, the microfluidic device for analysing a compound in aqueous phase has the following differences with regards to microfluidic devices for analysing a gaseous compound such as described hereinabove:
the gas pump 5 is replaced with a peristaltic pump (or a syringe pump) 3, preferentially par a peristaltic pump (or the syringe pump) 3 comprising at least one additional channel;
the ratio between the flow rate of the liquid phase comprising the compound to be analysed and the flow rate of the liquid phase comprising the derivative agent is between 0.01 and 100, preferentially between 0.1 and 10, further more preferentially between 0.5 and 2;
the length of the capillary tube 18 can vary between 25 cm and 3 m, preferentially between 50 cm and 2 m for a liquid flow rate of 10 μL/min and between 2.5 cm and 50 cm, preferentially between 5 cm and 25 cm for a liquid flow rate of 1 μl/min.

The carrying out of an annular flow in the trapping tube 18 is no longer required. In an embodiment wherein the trapping tube 18 is regulated in temperature, with the residence time in the trapping tube 18 being longer, the trapping tube 18 can advantageously be shortened while still allowing for the total derivatization reaction of the compound to be analysed. In an embodiment wherein the trapping tube is not regulated in temperature, the trapping tube can be suppressed. In an embodiment wherein it is not desired to modify the length of the trapping tube in relation to the microfluidic devices for analysing a gaseous compound such as described hereinabove, in order to be able to reuse said device as is in order to carry out an analyse of a compound in aqueous phase, an annular flow is carried out using an inert gas, devoid of reactive impurities that interfere with the measurement, such as for example pure nitrogen. In this embodiment, the two solutions (compound to be analysed in aqueous phase and solution comprising a derivative agent) are mixed upstream of the trapping tube: the residence time in the trapping tube and in fact the reaction time are then retained.

According to an embodiment, the means for eliminating the gaseous phase that appeared during the reaction between said compound and the derivative agent are such as those described hereinabove.

According to an embodiment, the means for determining the concentration of compound in aqueous phase are such as those described hereinabove. According to an embodiment, the detection limit of the compound in aqueous phase is 0.5 μg/L.

This invention also relates to a method for determining the concentration of a compound in aqueous phase using the device of this invention.

1-24. (canceled)

25. Microfluidic device for analysing a gaseous compound, in a dynamic manner and while flowing, said device comprising:
mixing means that make it possible to co-elute in a capillary tube a gaseous phase comprising said gaseous compound and a solution comprising a derivative agent;
means for reacting the gaseous compound in solution with the derivative agent;
means for eliminating the gaseous phase; and;
means for determining the concentration of a gaseous compound.

26. The device according to claim 25, wherein the mixing means that make it possible to co-elute in a capillary tube a gaseous phase comprising said gaseous compound and a solution comprising a derivative agent include a trapping cell comprising a capillary tube supplied with gaseous phase by a capillary gaseous phase supply tube and supplied with solution by a solution supply tube.

27. The device according to claim 25, wherein the means for reacting the gaseous compound in solution with the derivative agent include a means of thermoregulation that make it possible to regulate the temperature of the mixture between the solution comprising a derivative agent and the gaseous compound initially in gaseous phase trapped in the solution comprising a derivative agent.

28. The device according to claim 25, wherein the mixing means that make it possible to co-elute in a capillary tube a gaseous phase comprising said gaseous compound and a solution comprising a derivative agent and the means for reacting the gaseous compound in solution with the derivative agent are separated.

29. The device according to claim 25, wherein the mixing means that make it possible to co-elute in a capillary tube a gaseous phase comprising said gaseous compound and a solution comprising a derivative agent and the means for reacting the gaseous compound in solution with the derivative agent are combined.

30. The device according to claim 25, wherein the mixing means further comprise means for obtaining an annular flow, an annular-slug flow or a slug flow.

31. The device according to claim 25, wherein the mixing means further comprise means for obtaining an annular flow, an annular-slug flow or a slug flow, and wherein the means for obtaining an annular flow, an annular-slug flow or a slug flow include a mass flow regulator adapted for gases, a gas pump and a peristaltic pump or a syringe pump regulated in such a way that the gaseous flow rate is 100 to 10,000 times greater than the liquid flow rate.

32. The device according to claim 25, wherein the mixing means further comprise means for obtaining an annular flow, an annular-slug flow or a slug flow, and wherein the means for obtaining an annular flow include a mass flow regulator adapted for gases, a gas pump and a peristaltic pump or a syringe pump regulated in such a way that the gaseous flow rate is 850 to 10,000 times greater than the liquid flow rate.

33. The device according to claim 25, wherein the mixing means that make it possible to co-elute in a capillary tube a gaseous phase comprising said gaseous compound and a solution comprising a derivative agent include a trapping cell comprising a capillary tube supplied with gaseous phase by a capillary gaseous phase supply tube and supplied with solution by a solution supply tube, and wherein the capillary gaseous phase supply tube and the solution supply tube are perpendicular, concentric or oriented with respect to one another with an angle between 0° and 90°.

34. The device according to claim 25, wherein the mixing means that make it possible to co-elute in a capillary tube a gaseous phase comprising said gaseous compound and a solution comprising a derivative agent include a trapping cell comprising a capillary tube supplied with gaseous phase by a capillary gaseous phase supply tube and supplied with solution by a solution supply tube, and wherein the capillary tube, for the supply with gaseous phase is of a smaller outer diameter than the inner diameter of the capillary tube of the mixing means, with said capillary tube being inserted partially into the capillary tube of the mixing means.

35. The device according to claim 25, wherein the mixing means that make it possible to co-elute in a capillary tube a gaseous phase comprising said gaseous compound and a solution comprising a derivative agent include a trapping cell comprising a capillary tube supplied with gaseous phase by a capillary gaseous phase supply tube and supplied with solution by a solution supply tube, wherein the capillary tube, for the supply with gaseous phase is of a smaller outer diameter than the inner diameter of the capillary tube of the mixing means, with said capillary tube being inserted partially into the capillary tube of the mixing means, and further wherein the supplying with solution of the capillary tube of the mixing means by the solution supply tube is carried out upstream of the distal end of the capillary gaseous phase supply tube.

36. The device according to claim 25, wherein the means for determining the concentration of a gaseous compound include:
means for measuring the concentration of the derivative compound obtained from the reaction between the gaseous compound and the derivative agent; and;
means for calculating the concentration of a gaseous compound using the concentration of the derivative compound obtained hereinabove.

37. The device according to claim 25, wherein the means for determining the concentration of a gaseous compound include:
means for measuring the concentration of the derivative compound obtained from the reaction between the gaseous compound and the derivative agent; and;
means for calculating the concentration of a gaseous compound using the concentration of the derivative compound obtained hereinabove;
and further wherein the measurement of the concentration of the derivative compound is carried out by colorimetry.

38. The device according to claim 25, wherein the means for determining the concentration of a gaseous compound include:
means for measuring the concentration of the derivative compound obtained from the reaction between the gaseous compound and the derivative agent; and;
means for calculating the concentration of a gaseous compound using the concentration of the derivative compound obtained hereinabove;
and further wherein the means for measuring the concentration of the derivative compound include a wave guide, a light source and a detector.

39. The device according to claim 25, wherein the means for determining the concentration of a gaseous compound include:
means for measuring the concentration of the derivative compound obtained from the reaction between the gaseous compound and the derivative agent; and;
means for calculating the concentration of a gaseous compound using the concentration of the derivative compound obtained hereinabove;
and further wherein the measurement of the concentration of the derivative compound is carried out by fluorescence spectroscopy.

40. The device according to claim 25, wherein the means for determining the concentration of a gaseous compound include:
means for measuring the concentration of the derivative compound obtained from the reaction between the gaseous compound and the derivative agent; and;
means for calculating the concentration of a gaseous compound using the concentration of the derivative compound obtained hereinabove;
and further wherein the means for measuring the concentration of the derivative compound include a light source, a fluorescence cell comprising a wave guide and a detector.

41. The device according to claim 25, wherein the means for determining the concentration of a gaseous compound include:
means for measuring the concentration of the derivative compound obtained from the reaction between the gaseous compound and the derivative agent; and;
means for calculating the concentration of a gaseous compound using the concentration of the derivative compound obtained hereinabove;
and further wherein the measurement of the concentration of the derivative compound is carried out by fluorescence spectroscopy and by colorimetry.

42. Method for determining the concentration of a gaseous compound using a microfluidic device for analysing a gaseous compound, in a dynamic manner and while flowing, said device comprising:
mixing means that make it possible to co-elute in a capillary tube a gaseous phase comprising said gaseous compound and a solution comprising a derivative agent;
means for reacting the gaseous compound in solution with the derivative agent;
means for eliminating the gaseous phase; and
means for determining the concentration of a gaseous compound.

43. The method according to claim 42, wherein gaseous compound is formaldehyde.

44. The method according to claim 42, wherein the consumption of liquid derivative agent is less than 1 mL/min or the temporal resolution is less than 5 min.
